(19) United States
(12) Patent Application Publication
(10) Pub. No.: US 2005/0131446 A1
(43) Pub. Date: Jun. 16, 2005

(54) Catheter with protected occlusion balloon

(75) Inventors: Katherine G. Coughlin, Ipswich, MA (US); Michelle Provost-Tine, North Andover, MA (US)

Correspondence Address: Catherine C. Maresh;Medtronic Vascular, Inc., IP Legal, 3576 Unocal Place, Santa Rosa, CA, 95403, US

(73) Assignee: MEDTRONIC VASCULAR, INC.

(21) Appl. No.: 10/736,827

(22) Filed: Dec. 16, 2003

(51) Int. Cl. A61M 29/00

(52) U.S. Cl. 606/194

(57) ABSTRACT

A catheter for treating a vascular condition, the catheter including an elongate shaft, an inflatable occlusion balloon disposed on a distal region of the catheter and a stop member disposed on the catheter proximal the occlusion balloon. The stop member protects the occlusion balloon from damage by blocking the sliding advancement of a treatment instrument over the catheter shaft.

### FIELD OF THE INVENTION

This invention relates generally to catheter systems used in treatment of stenoses within blood vessels. More specifically, the invention relates to a catheter having a distal occlusion balloon that is protected against damage by a treatment instrument proximal to the occlusion balloon.

### BACKGROUND OF THE INVENTION

Human blood vessels often become narrowed or blocked by atherosclerotic plaque, thrombi or other deposits. Such stenoses reduce the blood-carrying capacity of the vessel and can cause serious and permanent injury, even death. When significant stenosis is detected, medical interventions are performed to prevent major adverse events such as myocardial infarction, stroke or death. Besides surgical modalities, there are less-invasive transluminal catheterization techniques, such as balloon angioplasty, atherectomy, deployment of stents and introduction of medication by infusion. These catheter-based treatments carry a risk of dislodging particles of the stenotic material, which can move downstream to cause an embolism. Thus, there is a need to contain and remove such embolic debris.

Systems of catheters and/or guidewires are used in the treatment of stenoses and emboli containment within blood vessels. Before moving an interventional catheter into a stenosis, a distal protection catheter can be advanced through the stenosis into a position such that an occlusion balloon can be inflated distal to the stenosis. The distal protection catheter typically also serves as a guidewire for a treatment catheter that slides there over. As described in U.S. Pat. No. 6,569,148, for example, an inflated distal occlusion balloon can block distal blood flow to prevent distal embolization by particulate debris entrained in the blood. Such occlusion balloons are thin-walled and fragile, and are subject to damage from treatment catheters approaching from the proximal side of the balloon. It is challenging to avoid such contact with occlusion balloons because catheterization procedures take place under the visual limitations of fluoroscopy, sometimes in the coronary arteries of a beating heart. The consequence of damage to an occlusion balloon may be that it leaks and deflates, releasing any captured embolic particles, thus defeating the purpose of the distal protection catheter. It is desirable to protect a distal occlusion balloon from being damaged by a treatment catheter approaching from the proximal side of the occlusion balloon.

### SUMMARY OF THE INVENTION

The present invention addresses the need to prevent treatment instruments from sliding forward into contact with the occlusion balloon of a distal protection catheter. It will be appreciated that, as used herein, the term “catheter” is broadly used to refer to a number of medical instruments, including without limitation, occlusion catheters or guidewires, therapy catheters and the like.

One aspect of the present invention provides a catheter for treating a vascular condition. The catheter includes an elongate catheter shaft, an occlusion balloon disposed on a distal region of the catheter and a stop member disposed on the catheter shaft proximal the occlusion balloon.

A second aspect of this invention provides a system for treating a vascular condition including an occlusion balloon catheter having a stop member disposed on the catheter proximal the occlusion balloon, and a treatment instrument.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings. The drawings are not to scale. In all the figures, like elements share like reference numbers.

### BRIEF DESCRIPTION OF THE DRAWINGS

FIG. 1 is a perspective illustration of a catheter having an uninflated protected occlusion balloon, in accordance with a first embodiment of the current invention;

FIG. 2 is a perspective illustration of a catheter having an inflated protected occlusion balloon, in accordance with the first embodiment of the current invention;

FIG. 3 is a perspective illustration of a catheter having an inflated protected occlusion balloon, in accordance with a second embodiment of the current invention;

FIG. 4 is a perspective illustration of a catheter having an uninflated protected occlusion balloon, in accordance with a third embodiment of the current invention;

FIG. 5 is a perspective illustration of a catheter having an inflated protected occlusion balloon, in accordance with the third embodiment of the current invention;

FIG. 6 is a perspective illustration of a catheter having an uninflated protected occlusion balloon, in accordance with a fourth embodiment of the current invention;

FIG. 7 is a perspective illustration of a catheter having an inflated protected occlusion balloon, in accordance with a fourth embodiment of the current invention;

FIG. 8 is a side view of a catheter having an uninflated protected occlusion balloon, in accordance with a fifth embodiment of the current invention;

FIG. 9 is a side view of a catheter having an inflated protected occlusion balloon, in accordance with a fifth embodiment of the current invention.

### DETAILED DESCRIPTION OF THE INVENTION

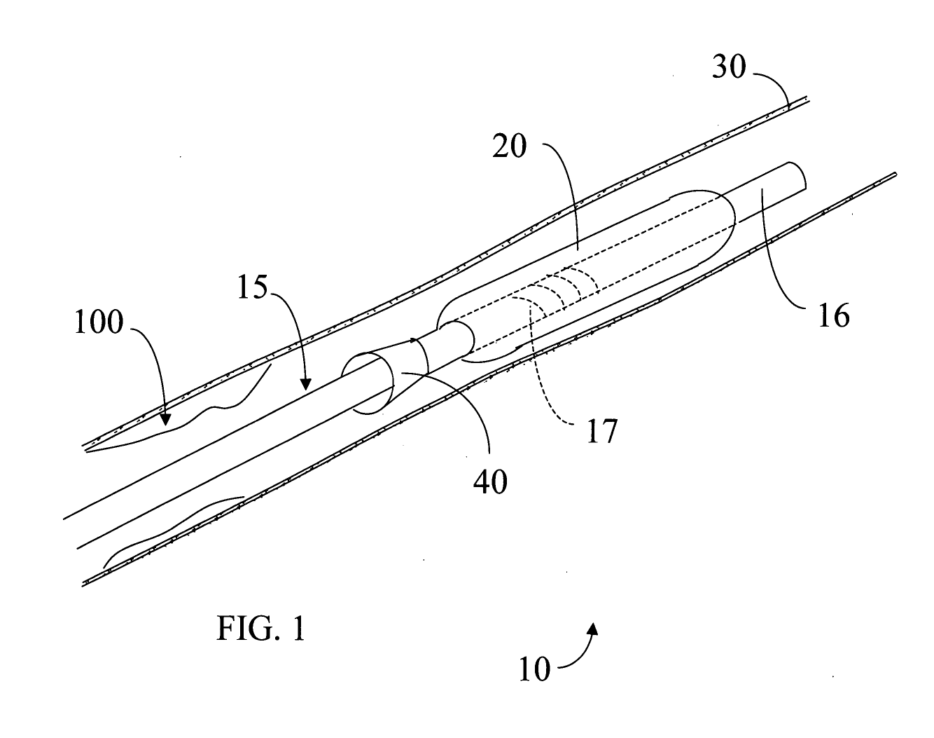

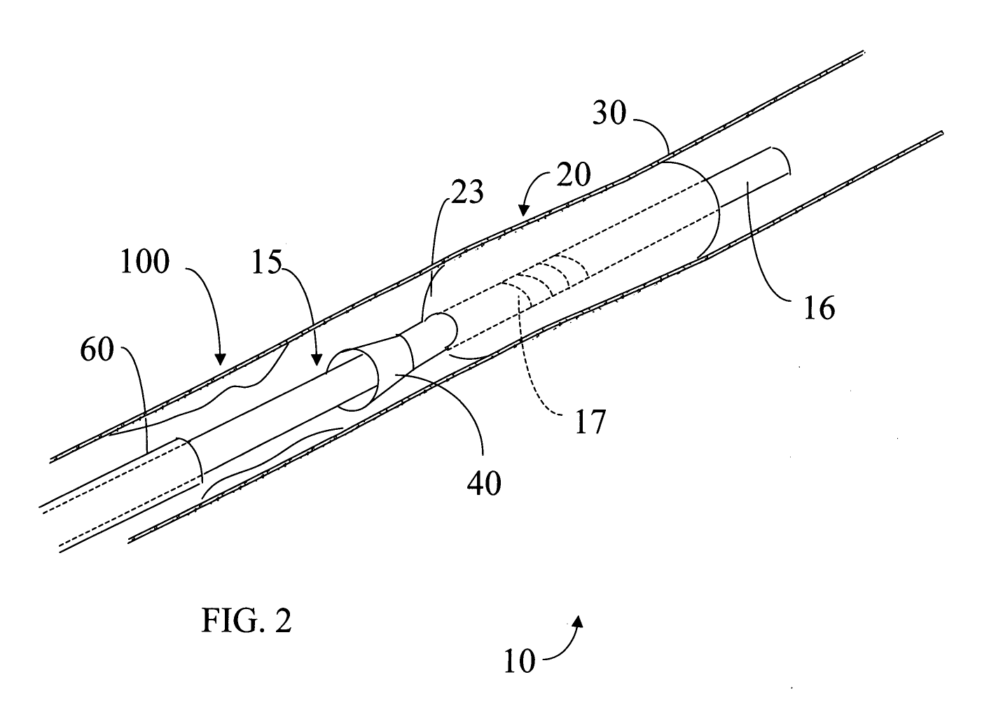

FIGS. 1 and 2 are perspective illustrations of system 10 for treating a vascular condition such as stenosis 100 in vessel 30, in accordance with a first embodiment of the current invention. System 10 includes catheter 15 having an inflation lumen (not shown) there through. Occlusion balloon 20 is mounted about distal region 16 of catheter 15 and is in fluid communication with the inflation lumen through one or more inflation ports, which may be round, oval, or helical in shape, such as spiral cut openings 17. The inflation lumen originates at the proximal end of catheter 15 and may terminate at the inflation port(s) or extend there beyond to a sealed distal end. Catheter 15 may also have a guidewire lumen extending end-to-end in an over-the-wire embodiment, or the guidewire lumen may extend through only relatively short distal region 16 in a rapid exchange embodiment.

FIG. 1 illustrates occlusion balloon 20 in an uninflated condition, wherein occlusion balloon 20 may be snugly collapsed about the shaft of catheter 15. Occlusion balloon 20 may be made from flexible biocompatible materials having a wide range of elasticity. Such materials may include thermoplastic elastomers, including styrenic TPEs such as styrene-ethylene-butylene-styrene (C-FLEX). Other suitable materials for occlusion balloon 20 are natural rubbers (latex), synthetic rubbers (silicone), or less elastic polymers such as polyesters, polyolefins, polyamides, polyvinyl chloride, and combinations of the above, such as block copolymers. If a relatively inelastic material is used to make occlusion balloon 20, its uninflated condition may include folds or “wings.” Conversely, an uninflated elastic balloon will collapse tightly about catheter 15 without any folds.

FIG. 2 illustrates occlusion balloon 20 inflated into apposition against the inner wall of vessel 30 to block the flow of blood and any embolic material entrained therein. When inflated without constraint, occlusion balloon 20 may be spherical or elongate in shape. Occlusion balloon 20 can be mounted to catheter 15 with adhesive, and with or without clamp rings, as will be understood to those of skill in the field of balloon catheters.

System 10 for treating a vascular condition also includes conical stop member 40 mounted about distal region 16 at a location proximal to occlusion balloon 20. Conical stop member 40 comprises a frustum of a cone with its base facing in the proximal direction (to the left in all figures). Conical stop member 40 may be a hollow rigid funnel, a hollow collapsible funnel or, a solid frustum of a cone. Conical stop member 40 may be spaced a relatively short distance from balloon proximal end 23, as shown in FIGS. 1 and 2. Alternatively, stop member 40 may be contiguous with proximal end 23 of occlusion balloon 20 to minimize the axial space along distal region 16 occupied by combined occlusion balloon 20 and stop member 40.

Conical stop member 40 may be formed of material selected from the group consisting of polyolefins, ethylene vinyl acetate (EVA), polyamides, polytetrafluoroethylene (PTFE), polyethylene terephthalate (PET), ionomer (SURLYN), polyethylene block amide copolymer (PEBA), and urethanes.

The shaft of catheter 15 may be formed of various polymeric materials commonly used for catheter construction. For catheter 15 to also be used as a guidewire for treatment catheters, it may preferably be formed of a hypotube comprising metal such as stainless steel or TiNi (nitinol), and it may also have a flexible distal tip such as a coil spring (not shown). Catheter 15 may also be provided with a coating on its outer surface. Slippery coatings may be hydrophilic or hydrophobic, such as TEFLON or a silicone composition. Antithrombogenic coatings such as heparin compounds may also be used. Suitable coatings and their application methods are well known in the art. Additional details relative to the catheter systems described herein may be found in U.S. Pat. No. 6,569,148.

During treatment of a patient, catheter 15 may be advanced through the patient's vasculature, such as vessel 30, until occlusion balloon 20 and stop member 40 are positioned distal to stenosis 100. As illustrated in FIG. 2, catheter 15 can perform the function of a guidewire to direct treatment instrument 60 into and across stenosis 100. As mentioned above, treatment instrument 60 may be an interventional catheter for angioplasty, atherectomy, stent deployment, infusion of medication, or aspiration of blood that may be contaminated with dislodged particles from stenosis 100. Before advancing treatment catheter 60 along catheter 15 into and through stenosis 100, occlusion balloon 20 is inflated by the delivery of a fluid through the inflation lumen of catheter 15 and inflation ports such as spiral cuts 17. The inflation fluid may be a dilute radiopaque contrast solution or CO₂ gas.

The annular space between catheter 15 and treatment instrument 60 may, optionally, be large enough to provide an intermediate pathway for irrigation, infusion of drugs or aspiration of the treatment area. Stop member 40 has an outer diameter large enough to prevent treatment instrument 60 from sliding forward into contact with occlusion balloon 20. If treatment instrument 60 slides along catheter 15 distally of stenosis 100, then conical stop member 40 stops the distal movement of treatment instrument 60 before it can contact, and possibly damage, occlusion balloon 20. If occlusion balloon 20 were to be damaged during treatment of stenosis 100, then occlusion balloon 20 could deflate unexpectedly, thus permitting the unplanned resumption of blood flow, which may carry any captured embolic particles downstream to embolize. During a normal treatment, static blood containing any dislodged particles is aspirated after use of treatment catheter 60. Then, occlusion balloon 20 is deflated to allow uncontaminated blood to begin flowing again, and system 10 is removed from the patient.

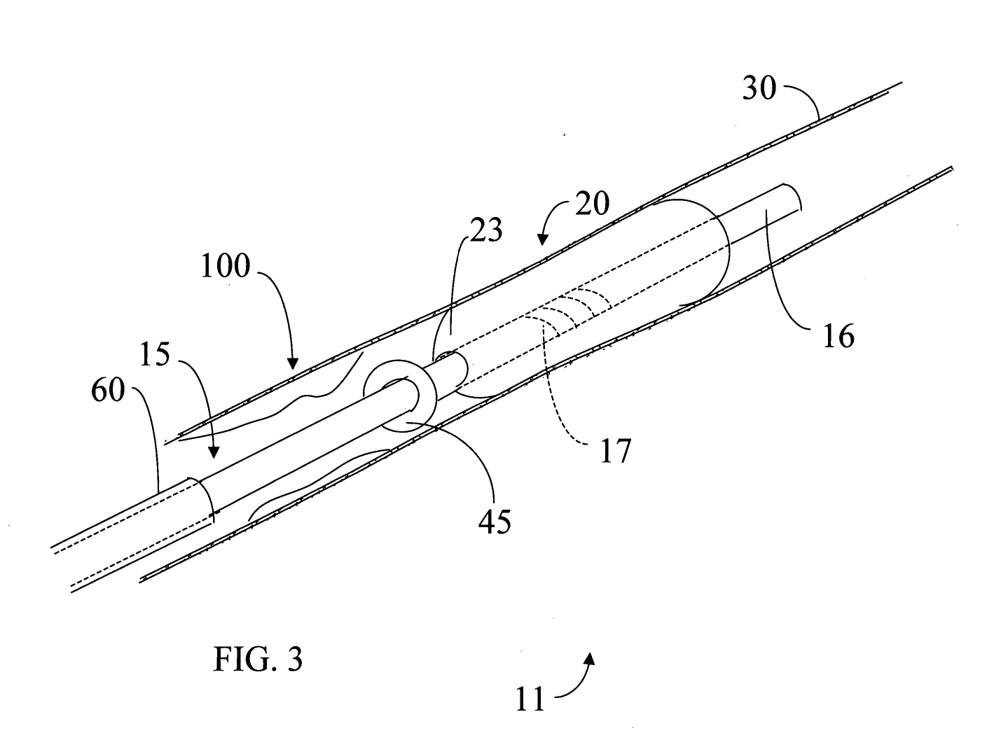

FIG. 3 is a perspective illustration of system 11 for treating a vascular condition, in accordance with a second embodiment of the current invention. In system 11, the stop member is stop ring 45 mounted on catheter 15 proximal to occlusion balloon 20. Stop ring 45 prevents treatment instrument 60 from sliding forward into contact with occlusion balloon 20. Stop ring 45 may be formed from biocompatible materials having sufficient hardness to resist deformation by an abutting treatment instrument 60. Examples of such materials include polyolefins, ethylene vinyl acetate (EVA), polyamides, polyesters, ionomers, PEBAX, urethanes, or metals that are radiopaque or radiolucent. Comparable to the first embodiment described above, stop ring 45 may be spaced a relatively short distance from balloon proximal end 23, as shown in FIG. 3, or stop ring 45 may be contiguous with proximal end 23 of occlusion balloon 20.

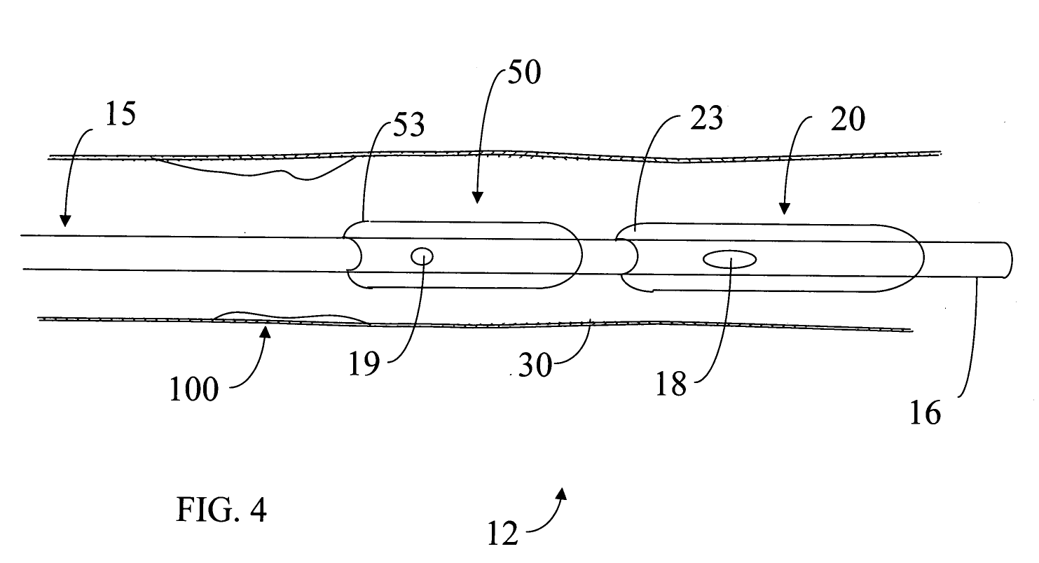

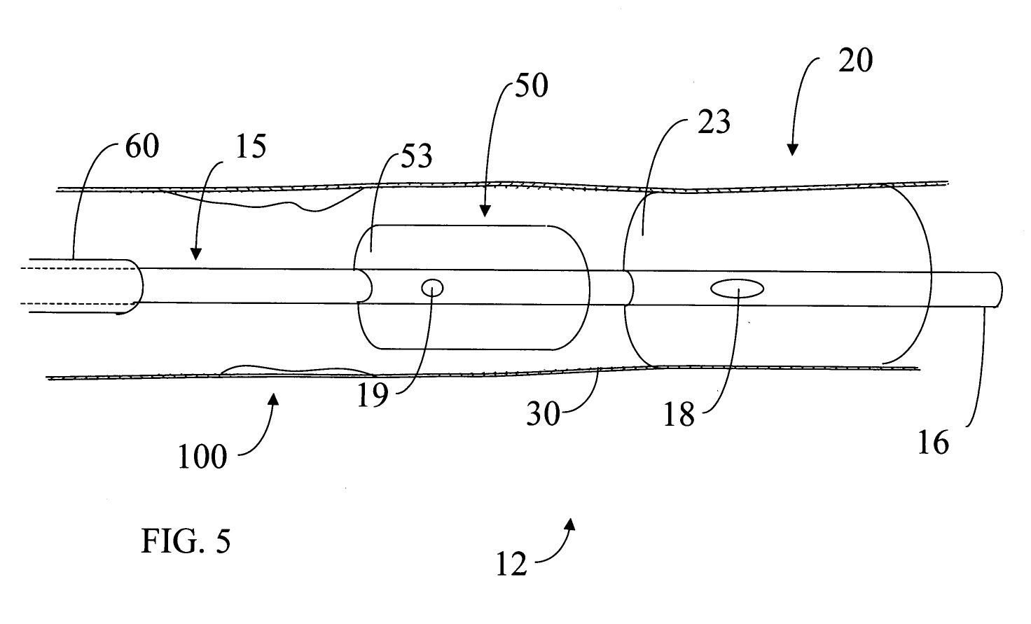

FIGS. 4 and 5 are perspective illustrations of system 12 for treating a vascular condition, in accordance with a third embodiment of the current invention. In system 12, the stop member is stop balloon 50 mounted around catheter 15 proximal to occlusion balloon 20. Stop balloon 50 is in fluid communication through first port 19 with an inflation lumen (not shown) extending through catheter 15. As in previously described embodiments, occlusion balloon 20 is in fluid communication via second port 18 with an inflation lumen through catheter 15. Catheter 15 may include separate inflation lumens, or occlusion balloon 20 and stop balloon 50 may be in fluid communication with, and may be inflated through, a single inflation lumen. FIG. 4 shows occlusion balloon 20 in an uninflated condition, and FIG. 5. shows occlusion balloon 20 in an inflated condition. When inflated, occlusion balloon 20 may be in apposition with the inner walls of vessels 30 as small as 2 to 4 mm in diameter, although the present invention can be made for use within larger vessels. Preferably, the uninflated profile of stop balloon 50 is no larger than the uninflated profile of occlusion balloon 20.

Stop balloon 50 is conveniently inflatable and deflatable, like occlusion balloon 20. However, by using thicker and/or stronger material, stop balloon 50 is relatively more resistant to damage from abutting treatment instruments 60. Stop balloon 50 may be formed from the same group of materials described above with respect to making occlusion balloon 20; Namely, thermoplastic elastomers, including styrenic TPEs such as styrene-ethylene-butylene-styrene (C-FLEX), natural rubbers (latex), synthetic rubbers (silicone), polyesters, polyolefins, polyamides, polyvinyl chloride, and combinations of the above, such as block copolymers.

During treatment of a patient, catheter 15 may be advanced through the patient's vasculature, such as vessel 30, until occlusion balloon 20 and stop balloon 50 are positioned distal to stenosis 100. As illustrated in FIG. 5 and comparable to system 10 above, catheter 15 can also perform the function of a guidewire to direct treatment instrument 60 into and across stenosis 100. Before advancing treatment catheter 60 into and through stenosis 100, occlusion balloon 20 and balloon stop 50 are inflated by the delivery of a fluid through one or more inflation lumens in catheter 15 and inflation ports 18 and 19, respectively. The inflation fluid may be a dilute radiopaque contrast solution or CO₂ gas. As discussed above, the annular space between catheter 15 and treatment instrument 60 may provide an intermediate pathway for irrigation, infusion of drugs or aspiration of the treatment area.

Balloon stop 50 has an inflated diameter large enough to prevent treatment instrument 60 from sliding forward into contact with occlusion balloon 20. To perform its stop function, inflated balloon stop 50 does not need to contact the inner wall of vessel 30, although such contact may occur, especially in tortuous vessels. Thus, the inflated diameter of balloon stop 50 is typically smaller than the inflated diameter of occlusion balloon 20. The inflated shape of stop balloon 50 may be spherical or elongate. In one embodiment, stop balloon 50 may have an inflated diameter of about 0.020 inches (0.51 mm) and a length of about 2 mm with proximal and distal ends of stop balloon 50 each being affixed to the shaft of catheter 15 along a length of approximately 1 mm. Stop balloon 50 can be mounted to catheter 15 with adhesive, and with or without clamp rings, as will be understood to those of skill in the field of balloon catheters. Comparable to the embodiments described above, balloon stop 50 may be spaced a relatively short distance from balloon proximal end 23, as shown in FIGS. 4 and 5, or balloon stop member 55 may be contiguous with proximal end 23 of occlusion balloon 20, as will be described below.

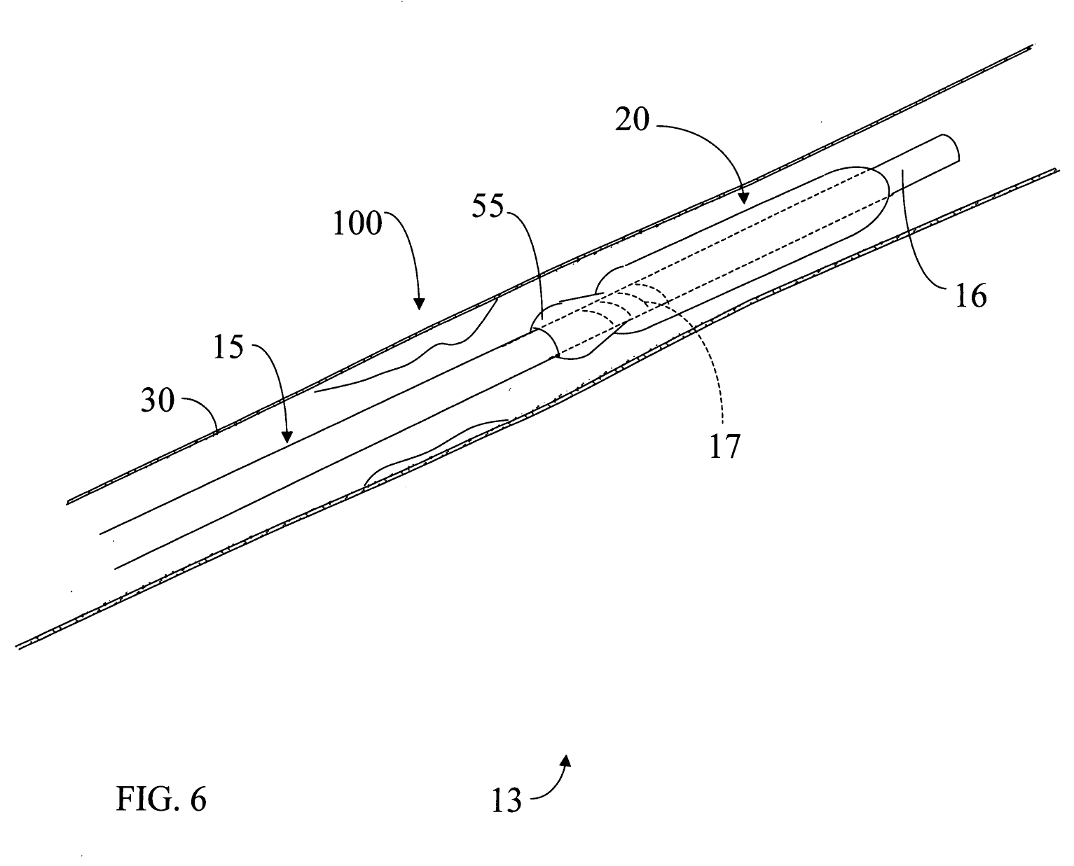

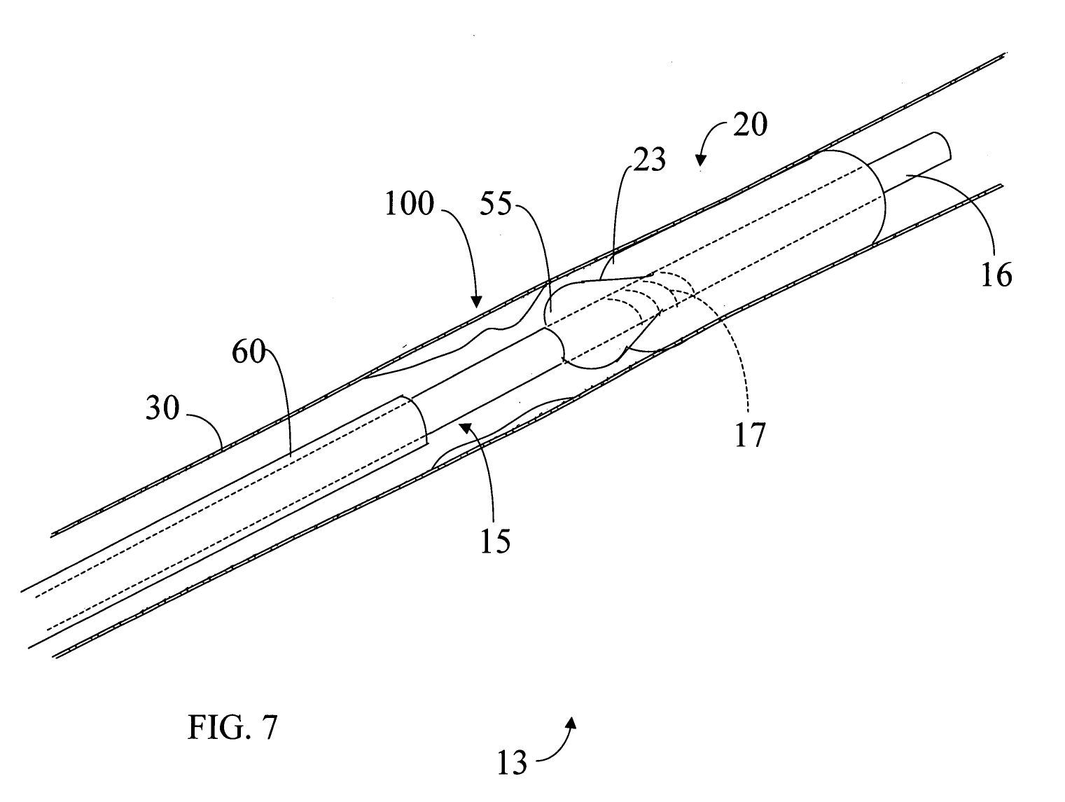

FIGS. 6 and 7 are perspective illustrations of system 13 for treating a vascular condition, in accordance with a fourth embodiment of the current invention. In system 13, the stop member is stop balloon 55 mounted around catheter 15 proximal to occlusion balloon 20. Proximal end 23 of occlusion balloon 20 and distal end 55 of stop balloon 50 may overlap each other. Alternatively, stop balloon 55 and occlusion balloon 20 may be formed integrally, wherein stop balloon 55 has a relatively greater wall thickness of the same material. When inflated, such an integral balloon arrangement may optionally have a narrowed waist region between stop balloon 55 and occlusion balloon 20. The inflation lumen (not shown) of catheter 15 communicates with both stop balloon 50 and occlusion balloon 20 through spiral cut openings 17. Thus, both stop balloon 50 and occlusion balloon 20 are inflated simultaneously upon delivery of a fluid through the inflation lumen of catheter 15. The fluid may be a dilute radiopaque contrast solution or CO₂ gas. In an alternative embodiment (not shown), second port 18 under occlusion balloon 20 and first port 19 under stop balloon 55 may be used instead of spiral cut openings 17. As in previously described embodiments of the invention, stop balloon 55 prevents treatment instrument 60 from sliding forward into contact with occlusion balloon 20.

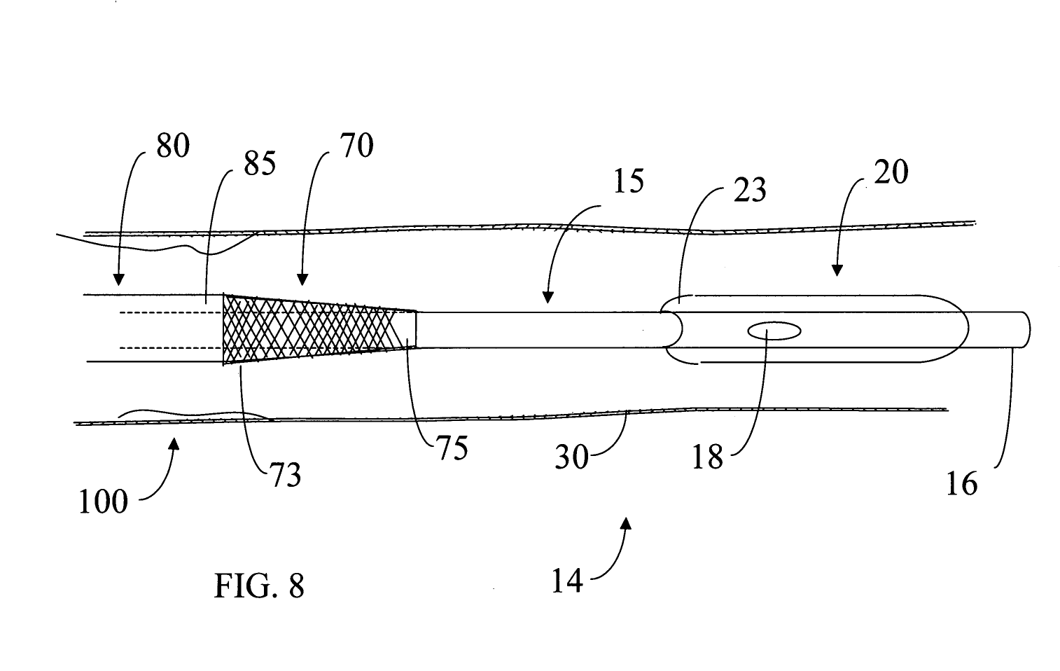

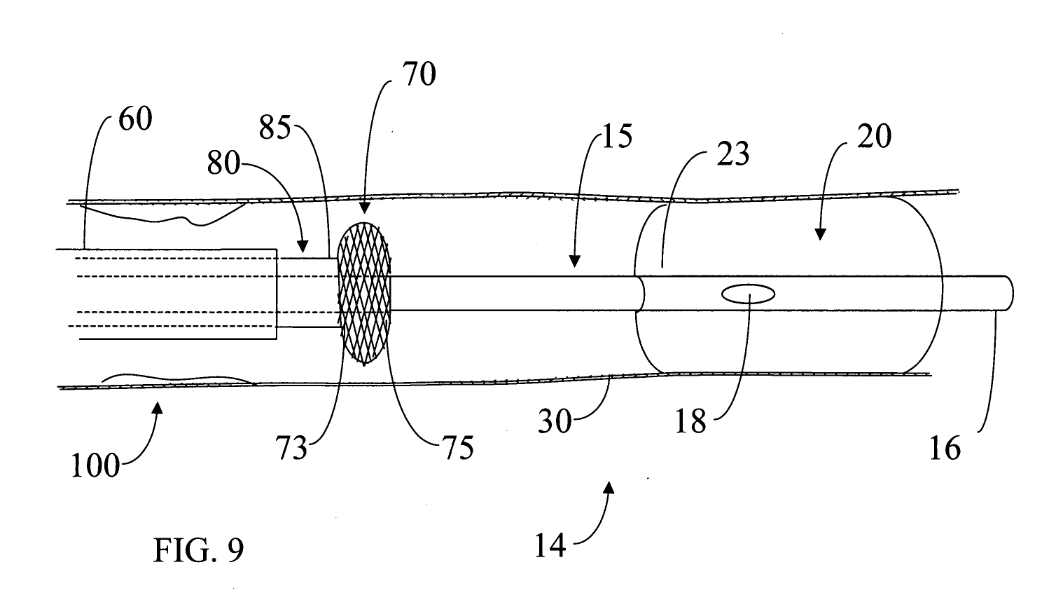

FIGS. 8 and 9 are perspective illustrations of system 14 for treating a vascular condition, in accordance with a fifth embodiment of the current invention. In system 14, the stop member is mesh stop 70 positioned on catheter 15 proximal to occlusion balloon 20. Distal end 75 of mesh stop 70 is coupled to distal region 16 of catheter 15. Proximal end 73 of mesh stop 70 is coupled to distal end 85 of actuator sheath 80, which is sized and shaped to be slidingly disposed about the shaft of catheter 15 to operate mesh stop 70, as described below. Actuator sheath 80 may be formed from suitable biocompatible tubing such as thermoset polyimide, or metal hypotubing.

Mesh stop 70 may be formed from braided metal wires such as TiNi (nitinol) or stainless steel, or from braided polymeric filaments. Ends 73, 75 of mesh stop 70 may be attached to catheter 15 and to actuator sheath 80, respectively, by solder, adhesive or by mechanical attachments such as crimp bands. In response to relative sliding movement between actuator sheath 80 and the shaft of catheter 15, mesh stop 70 is transformable between the collapsed configuration shown in FIG. 8 and the expanded configuration shown in FIG. 9. Moving of the ends of mesh stop 70 apart or towards each other causes the tubular braid to collapse or expand, respectively, as will be understood by one of skill in the art. Preferably, the collapsed diameter of mesh stop 70 is no larger than the deflated profile of occlusion balloon 20.

During treatment of a patient, catheter 15 and actuator sheath 80 may be advanced through the patient's vasculature, such as vessel 30, until occlusion balloon 20 and mesh stop 70 are positioned distal to stenosis 100. As illustrated in FIG. 9 and as described above, catheter 15 and surrounding actuator sheath 80 can perform the function of a guidewire to direct treatment instrument 60 into and across stenosis 100. Before advancing treatment catheter 60 into and through stenosis 100, occlusion balloon 20 is inflated by the delivery of a fluid through an inflation lumen in catheter 15 and second inflation port 18.

Additionally, prior to advancing treatment catheter 60 into and through stenosis 100, mesh stop 70 is expanded by pushing actuator sheath 80 distally while pulling catheter 15 proximally, causing the diameter of mesh stop 70 to increase as the length of mesh stop 70 decreases. To protect occlusion balloon 20, the expanded diameter of mesh stop 70 needs to be only large enough to block advancement of treatment instrument 60. Thus, the expanded diameter of mesh stop 70 does not need to contact the inner wall of vessel 30, although such contact may occur, especially in tortuous vessels. Once mesh stop 70 is expanded, sheath 80 may be temporarily held in position with respect to catheter 15 by a friction mechanism (not shown). The friction mechanism may include slight distortions or wave-like bends along a section of catheter 15. Sheath 80 fits closely around catheter 15 so the slight bends can provide sufficient normal force to hold the two parts in a fixed relative position until the clinician collapses mesh stop 70 by pulling actuator sheath 80 proximally while pushing catheter 15 distally. Actuator sheath 80 may also be held in place by an external mechanical locking mechanism (not shown) provided at the proximal end of catheter 15. Both mesh stop 70 and occluder balloon 20 are collapsed to remove system 14 from the patient.

As in the other embodiments of the current invention described above, mesh stop 70 prevents treatment instrument 60 from sliding distally over catheter 15 and actuator sheath 80 into contact with occlusion balloon 20. Thus, the fragile occlusion balloon 20 is not damaged. Mesh stop 70 may be spaced a relatively short distance from balloon proximal end 23, as shown in FIGS. 9 and 10, or mesh stop 70 may be contiguous with proximal end 23 of occlusion balloon 20.

Although the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A catheter for treating a vascular condition, the catheter comprising:
an elongate catheter shaft having a lumen and a distal region;
an occlusion balloon disposed about the distal region and being in fluid communication with the lumen; and
a stop member disposed on the catheter shaft proximal the occlusion balloon.

2. The catheter of claim 1, wherein the stop member has an outer diameter large enough to prevent a treatment instrument from sliding on the catheter shaft distally beyond the stop member.

3. The catheter of claim 1, wherein the stop member comprises a frustum of a cone.

4. The catheter of claim 3, wherein the frustum is hollow.

5. The catheter of claim 1, wherein the catheter shaft comprises a hypotube.

6. The catheter of claim 1, wherein the stop member comprises a stop ring.

7. The catheter of claim 1 wherein the stop member comprises material selected from the group consisting of polyolefins, ethylene vinyl acetate (EVA), polyamides, polyesters, ionomers, PEBAX, urethanes, and metals.

8. The catheter of claim 1, wherein the stop member comprises a stop balloon mounted around the catheter shaft and being in fluid communication with the lumen.

9. The catheter of claim 8 wherein the stop balloon is contiguous with the occlusion balloon.

10. The catheter of claim 8 wherein the stop balloon and the occlusion balloon are formed integrally and wherein the stop balloon has a thickness that is greater than a thickness of the occlusion balloon.

11. The catheter of claim 8 wherein the stop balloon comprises a material selected from the group consisting of thermoplastic elastomers, styrenic TPEs, natural rubbers, synthetic rubbers, polyesters, polyolefins, polyamides, polyvinyl chloride, and combinations of the above, such as block copolymers.

12. The catheter of claim 8 wherein the stop balloon has an inflated diameter smaller than an inflated diameter of the occlusion balloon.

13. The catheter of claim 1, further comprising:
an elongate actuator sheath slidably disposed about the catheter shaft, and
wherein the stop member comprises a mesh stop having a distal end coupled to the distal region of the catheter shaft and a proximal end coupled to the actuator sheath such that the mesh stop is transformable between a collapsed configuration and an expanded configuration in response to relative sliding movement of the actuator sheath and the catheter shaft.

14. The catheter of claim 13 wherein the mesh stop comprises braided metal wires.

15. The catheter of claim 13 wherein the mesh stop comprises braided plastic filaments.

16. A system for treating a vascular condition comprising:
an elongate catheter shaft having a lumen and a distal region;
an occlusion balloon disposed about the distal region and being in fluid communication with the lumen;
a stop member disposed on the catheter shaft proximal the occlusion balloon; and
a treatment instrument slidably mountable over the catheter shaft.

17. The system of claim 16 wherein the stop member and the treatment instrument are each sized and shaped such that the treatment instrument cannot slide distally beyond the stop member.

18. The system of claim 16, wherein the treatment instrument is selected from a group consisting of catheters for angioplasty, atherectomy, stent deployment, infusion of medication, or aspiration of blood.
